(19) United States
(12) Patent Application Publication
(10) Pub. No.: US 2021/0128667 A1
(43) Pub. Date: May 6, 2021

(54) IMMUNOPROTEASOME INHIBITORS AND IMMUNOSUPPRESSIVE AGENT IN THE TREATMENT OF AUTOIMMUNE DISORDERS

(71) Applicant: KEZAR LIFE SCIENCES, South San Francisco, CA (US)

(72) Inventor: Tony Muchamuel, Boulder Creek, CA (US)

(21) Appl. No.: 16/640,754

(22) PCT Filed: Aug. 23, 2018
(86) PCT No.: PCT/US2018/047622

§ 371 Date: Feb. 21, 2020

Related U.S. Application Data
(60) Provisional application No. 62/549,020, filed on Aug. 23, 2017.

(51) Int. Cl. A61K 38/06; A61K 31/5377; A61K 31/4706; A61K 31/52; A61K 31/675

(52) U.S. Cl. CPC A61K 38/06; A61K 31/5377; A61K 31/675; A61K 31/52; A61K 31/4706

(57) ABSTRACT

Provided herein are methods of treating autoimmune diseases comprising administering to a subject suffering therefrom an immunoproteasome inhibitor and an immunosuppressive agent.

### BACKGROUND

In eukaryotes, protein degradation is predominately mediated through the ubiquitin proteasome pathway in which proteins targeted for destruction are ligated to the 76 amino acid polypeptide ubiquitin. Once targeted, ubiquitinated proteins then serve as substrates for the 26S proteasome, a multicatalytic protease, which cleaves proteins into short peptides through the action of its three major proteolytic activities. While having a general function in intracellular protein turnover, proteasome-mediated degradation also plays a key role in many processes such as major histocompatibility complex (MHC) class I antigen presentation, apoptosis, cell growth regulation, NF-κB activation, antigen processing, and transduction of pro-inflammatory signals.

The 20S proteasome is a 700 kDa cylindrical-shaped multicatalytic protease complex comprised of 28 subunits organized into four rings. In yeast and other eukaryotes, 7 different α subunits form the outer rings and 7 different β subunits comprise the inner rings. The a subunits serve as binding sites for the 19S (PA700) and 11S (PA28) regulatory complexes, as well as a physical barrier for the inner proteolytic chamber formed by the two 7 subunit rings. Thus, in vivo, the proteasome is believed to exist as a 26S particle (“the 26S proteasome”). In vivo experiments have shown that inhibition of the 20S form of the proteasome can be readily correlated to inhibition of 26S proteasome. Cleavage of amino-terminal prosequences of active site β subunits during particle formation expose amino-terminal threonine residues, which serve as the catalytic nucleophiles. The subunits responsible for catalytic activity in proteasomes thus possess an amino terminal nucleophilic residue, and these subunits belong to the family of N-terminal nucleophile (Ntn) hydrolases (where the nucleophilic N-terminal residue is, for example, Cys, Ser, Thr, and other nucleophilic moieties). This family includes, for example, penicillin G acylase (PGA), penicillin V acylase (PVA), glutamine PRPP amidotransferase (GAT), and bacterial glycosylasparaginase. Through the use of different peptide substrates, three major proteolytic activities have been defined for the eukaryote 20S proteasome: chymotrypsin-like activity (CT-L), which cleaves after large hydrophobic residues; trypsin-like activity (T-L), which cleaves after basic residues; and peptidylglutamyl peptide hydrolyzing activity (PGPH) or caspase-like (C-L), which cleaves after acidic residues. In mammals, most cells and tissues express a “constitutive proteasome” in which the 3 active sites are β5, β1, and β2, which encode CT-L, C-L, and T-L activities, respectively. Higher order vertebrates also possess three interferon-γ-inducible β subunits (LMP7, LMP2 and MECL1), which replace their constitutive proteasome counterparts, β5, β1 and β2 respectively, thus altering the catalytic activities of the proteasome. The major proteasome proteolytic activities appear to be contributed by different catalytic sites, since inhibitors, point mutations in β subunits and the exchange of γ interferon-inducing β subunits alter these activities to various degrees.

### SUMMARY

Provided herein are methods of treating a subject suffering from an autoimmune disease comprising administering to the subject (a) an immunoproteasome inhibitor and (b) an immunosuppressive agent in an amount sufficient to treat the autoimmune disease. In various cases, the subject is human. In some cases, the autoimmune disease is lupus nephritis or systemic lupus erythematosus (SLE). In some cases, the autoimmune disease is systemic vasculitis or an idiopathic inflammatory myopathy.

In various cases, the immunoproteasome inhibitor and the immunosuppressive agent are administered simultaneously, and in some cases, can be co-formulated. In some cases, the immunoproteasome inhibitor and the immunosuppressive agent are administered sequentially (e.g., the immunoproteasome inhibitor before or after the immunosuppressive agent).

In various cases, the efficacy of administering the immunoproteasome inhibitor and the immunosuppressive agent is greater than the efficacy of administering the immunoproteasome inhibitor or the immunosuppressive agent alone. In various cases, the efficacy is exhibited by a decrease in proteinuria or urine protein to creatinine ratio compared to either (a) a subject not administered the immunoproteasome inhibitor and the immunosuppressive agent or (b) the same subject prior to administration of the immunoproteasome inhibitor and the immunosuppressive agent. In various cases, the subject exhibits a decrease in the urine protein to creatinine ratio of at least 50% compared to the urine protein to creatinine ratio of the subject prior to administration of the immunoproteasome inhibitor and the immunosuppressive agent. In various cases, the subject exhibits a urine protein to creatinine ratio of 0.5 or less after administration of the immunoproteasome inhibitor and the immunosuppressive agent.

In some cases, the immunoproteasome inhibitor has a structure of formula (I):

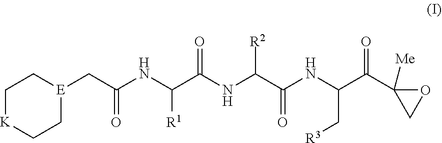

wherein K is CH(OH) or 0; E is N or CH; R¹ is CH₃, CH₂(OH)CH₃, or CH₂CN; R² is

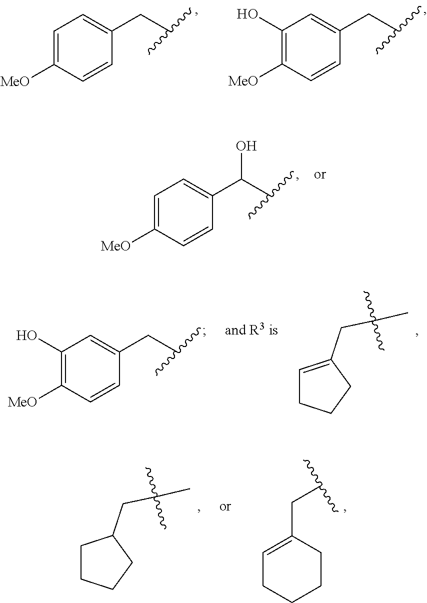

or a pharmaceutically acceptable salt thereof. In some cases, the immunoproteasome inhibitor has a structure of

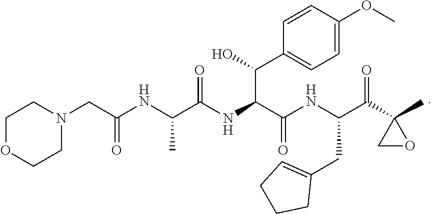 [SMILES: COc1ccc([C@@H](O)[C@H](NC(=O)[C@H](C)NC(=O)CN2CCOCC2)C(=O)N[C@@H](CC2=CCCC2)C(=O)[C@@]2(C)CO2)cc1]

or a pharmaceutically acceptable salt thereof. In various cases, the immunoproteasome inhibitor is administered in an amount of 1-300 mg per day. In various cases, the immunoproteasome inhibitor is administered in an amount of 40-120 mg per day. In various cases, the immunoproteasome inhibitor is administered orally, subcutaneously, topically, or intravenously, preferably subcutaneously. In various cases, the immunoproteasome inhibitor is administered once every 7 to 15 days, preferably once every 7 days.

In various cases, the immunosuppressive agent comprises a corticosteriod, an anti-miotic agent, a cytokine antagonist, a B-cell depleting agent, a nonsteriodal anti-inflammatory agent, or an antimalarial agent. In some cases, the immunosuppressive agent comprises one or more of aspirin, prednisone, methylprednisolone, sulfasalazine, leflunomide, hydroxychloroquine, belimumab, mycophenolate mofetil, mycophenolic acid, azathioprine, rituximab, ocrezilumab, entanercept, adalimumab, tocilizumab, tofacitinib, baracitinib, cyclosporine, cyclophosphamide, and tacrolimus. In some cases, the immunosuppressive agent is administered orally, subcutaneously, topically, or intravenously.

In some cases, the immunosuppressive agent comprises mycophenolate mofetil, mycophenolic acid, or a pharmaceutically acceptable salt thereof. In such cases, the mycophenolate mofetil, or pharmaceutically acceptable salt thereof, can be administered in an amount of 0.5-3 g per day, based upon mycophenolate mofetil weight, or the mycophenolic acid, or pharmaceutically acceptable slat thereof, is administered in an amount of 700 mg to 1500 mg per day, based upon mycophenolic acid weight. In such cases, mycophenolate mofetil, mycophenolic acid, or a pharmaceutically acceptable salt thereof, can be administerd once per day or twice per day.

In some cases, the immunosuppressive agent is hydroxychloroquine, azathioprine, or cyclophosphamide, or a pharmaceutically acceptable salt thereof. In some cases, the hydroxychloroquine, or pharmaceutically acceptable salt thereof, is administered in an amount of 150 to 325 mg per day, based upon hydroxychloroquine weight. In some cases, the azathioprine, or pharmaceutically acceptable salt thereof, is administered in an amount of 1 to 4 mg/kg per day, based upon azathioprine weight. In some cases, the cyclophosphamide, or pharmaceutically acceptable salt thereof, is administered in an amount of 500 to 1000 mg/m² every two to four weeks, based upon cyclophosphamide weight.

### BRIEF DESCRIPTION OF THE FIGURES

FIG. 1 shows the total proteinuria scores for mice given vehicle (circle), 5 mg/kg KZR-616 subcutaneously once weekly (square), 30 mg/kg mycophenolate mofetil (MMF) orally once daily (up triangle), or 5 mg/kg KZR-616 subcutaneously once weekly and 30 mg/kg orally MMF once daily (down triangle) over 25-35 weeks. The top right figure shows the prevention of severe proteinuria of these therapies and the bottom left shows mouse survival over 24-36 weeks when given these therapies.

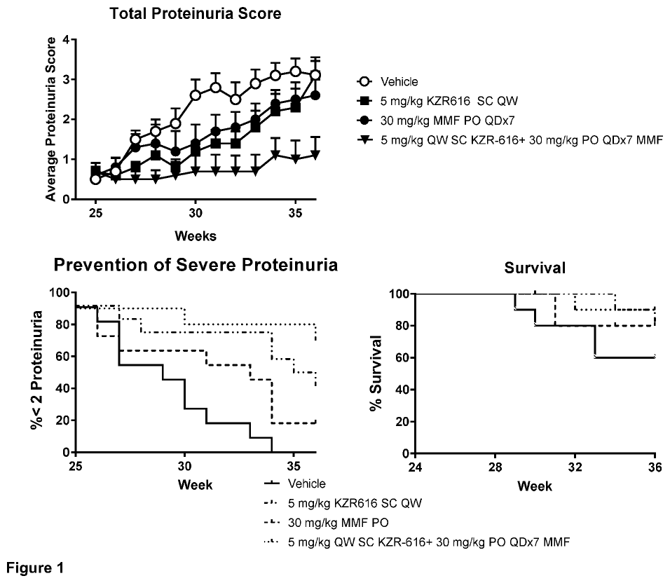

### DETAILED DESCRIPTION

Provided herein are methods of treating a subject suffering from an autoimmune disease comprising administering a combination therapy of an immunoproteasome inhibitor and an immunosuppressive agent in an amount sufficient to treat the autoimmune disease. The immunoproteasome inhibitor and/or the immunosuppressive agent may be present as a pharmaceutically acceptable salt thereof. The term “pharmaceutically acceptable salt” refers to the relatively non-toxic, inorganic or organic acid addition salt of a compound provided herein. These salts can be prepared in situ during the final isolation and purification of a compound provided herein, or by separately reacting the compound in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66: 1-19.)

The immunoproteasome inhibitor and the immunosuppressive agent can be administered at the same time or separately. In some cases when they are administered at the same time, the two agents are co-formulated. In cases when they are administered separately, the immunosuppressive agent is administered before the immunoproteasome inhibitor. In other cases when they are administered separately, the immunosuppressive agent is administered after the immunoproteasome inhibitor.

The methods disclosed herein can result in a decrease in proteinuria or urine protein to creatinine ratio compared to either (a) a subject not administered the immunoproteasome inhibitor and the immunosuppressive agent or (b) the same subject prior to administration of the immunoproteasome inhibitor and the immunosuppressive agent. Measurement of proteinuria or urine protein to creatinine ratio can be by any means known in the art. In some cases, the subject exhibits a decrease in the urine protein to creatinine ratio of at least 50% compared to the urine protein to creatinine ratio of the subject prior to administration of the immunoproteasome inhibitor and the immunosuppressive agent. in some cases, the subject exhibits a decrease in the urine protein to creatinine ratio of at least 55%, at least 60%, at least 65%, at least 70%, at least 75%, at least 80% , or at least 85% compared to the urine protein to creatinine ratio of the subject prior to administration of the immunoproteasome inhibitor and the immunosuppressive agent. In some cases, the subject exhibits a urine protein to creatinine ratio of 0.5 or less after administration of the immunoproteasome inhibitor and the immunosuppressive agent. In some case, the ratio is 0.4 or less, 0.35 or less, 0.3 or less, 0.3 or less, 0.25 or less, 0.2 or less, 0.15 or less, or 0.1 or less.

### Autoimmune Diseases

The methods provided herein are useful in the treatment of autoimmune diseases. An “autoimmune disease” as used herein is a disease or disorder arising from and directed against an individual's own tissues. Examples of autoimmune diseases include, but are not limited to, inflammatory responses such as inflammatory skin diseases including psoriasis and dermatitis (e.g., atopic dermatitis); systemic scleroderma and sclerosis; responses associated with inflammatory bowel disease (such as Crohn's disease and ulcerative colitis); respiratory distress syndrome (including adult respiratory distress syndrome(ARDS)); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE); diabetes mellitus (e.g., Type I diabetes mellitus or insulin dependent diabetes mellitus); multiple sclerosis; Reynaud's syndrome; autoimmune thyroiditis; allergic encephalomyelitis; Sjorgen's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Beheet disease; giant cell arteritis; immune complex nephritis; IgA nephropathy; IgM polyneuropathies; immune thrombocytopenic purpura (ITP) or autoimmune thrombocytopenia. In specific cases, the autoimmune disease is systemic lupus erythematosus or lupus nephritis. In some cases, the autoimmune disease is systemic vasculitis or idiopathic inflammatory myopathy.

Systemic lupus erythematosus (SLE) is a complex multi-organ autoimmune disease that is characterized by the development of a wide variety of autoantibodies, especially to components of the nucleus, specifically to DNA, RNA, and histones, in addition to red blood cells, platelets, serum proteins, and phospholipids.

SLE affects young adults, occurs more frequently in females than males (9:1 ratio), and is more common in African American, African Caribbean, Hispanic, and Asian populations (approximately 200 cases per 100,000) than in Caucasians (approximately 40 cases per 100,000). It is estimated that there are approximately 250,000 patients with SLE in the US (Feldman et al., 2013; Helmick et al., 2008).

Clinical manifestations range from relatively mild skin rashes and arthritis to glomerulonephritis, antibody mediated hemolytic anemia and thrombocytopenia, vasculitis, cardiac disease, and central nervous system disorders including seizures, psychosis, and cerebral vascular accidents (Wallace, 2015) (Tsokos, 2011). The accurate diagnosis of SLE can be difficult because the clinical manifestations vary considerably between patients, and the individual signs and symptoms of SLE can have multiple etiologies. Classification criteria have been developed by the American College of Rheumatology (ACR) (Hochberg, 1997; Tsokos, 2011).

SLE is thought to be the result of dysfunction of multiple components of the immune system, including defective clearance of apoptotic cellular components, a break in T-cell tolerance induction, and generation of antibodies against nuclear antigens (ANA) such as anti-double stranded DNA (anti-dsDNA) (Kaul et al., 2016). These ANA complex with antigens to create antigen antibody (Ag-Ab) complexes, which are deposited in various tissues and initiate inflammatory reactions via complement activation (e.g., arthritis and glomerulonephritis) or Type II hypersensitivity reactions in which antibodies directly target host cells and activate immune effector mechanisms that lead to phagocytosis (e.g., hemolytic anemia or immune thrombocytopenia). These inflammatory reactions lead to excessive complement activation, secretion of inflammatory cytokines, and activation of macrophages and neutrophils.

There is no cure for SLE. Treatment is targeted at controlling inflammation with a variety of anti-inflammatory and immunosuppresive agents including glucocorticosteroids, aspirin, other nonsteroidal anti-inflammatory agents (NSAIDs), and antimalarials (Hahn, 2011). Of the 3 types of treatments approved for SLE, NSAIDs were approved in 1948; hydroxychloroquine and corticosteroids were approved in 1955; and belimumab, a monoclonal antibody targeting the B-cell activating factor (BAFF), was approved in 2011(Lamore, Parmar, Patel, & Hilas, 2012).

Lupus nephritis (LN) is one of the most serious complications of SLE. LN, characterized by the presence of proteinuria >1 g/day and an active urinary sediment (hematuria, pyuria, casts) develops in about 50% of patients within 10 years of the initial diagnosis of SLE (Bertsias et al., 2012); (EMA Draft Guideline February 2015). LN is associated with considerable morbidity, including an increased risk of end-stage renal disease requiring dialysis or renal transplantation and an increased risk of death. The prevalence of LN is approximately 74,000 in the US and varies by race, occurring in approximately 20% of Caucasians and up to 60% of Blacks, Hispanics, and Asians with SLE (Feldman et al., 2013; Fernandez et al., 2007; Seligman, Lum, Olson, Li, & Criswell, 2002).

LN results when Ag-Ab complexes (predominantly DNA-anti-DNA) are deposited in the glomerular mesangium and glomerular basement membrane and activate serum complement. The resulting inflammatory response causes damage to the glomerular epithelium with loss of function. It is often accompanied by mesangial proliferation and subsequent sclerosis of the glomeruli. Histopathologically, LN can take many forms, ranging from a normal glomerular architecture with Ag-Ab complexes identified by immunofluorescence, to proliferative glomerulonephritis or wide-spread sclerosis of the glomeruli associated with end stage renal disease. The proliferative and membranous forms of glomerulonephritis are most frequently associated with proteinuria, which often reaches nephrotic levels. LN is classified according to the 2003 International Society of Nephrology/Renal Pathology Society (ISN/RPS) classification (Weening et al., 2004).

Approximately 50% of patients respond to these treatment regimens with improvement in proteinuria, but only about 25% attain a complete renal response (CRR) that is frequently defined as normalization of proteinuria and stabilization or improvement in serum creatinine, after 1 year of treatment (Rovin et al., 2012; Wofsy, Hillson, & Diamond, 2012). Attainment of CRR leads to a dramatic decrease in risk of end-stage renal disease (Chen, Korbet, Katz, Schwartz, & Lewis, 2008). Thus, approximately 75% of patients with LN have a sub-optimal response to induction therapy. These patients may subsequently receive treatment with a variety of alternative immunosuppressive or experimental agents, including rituximab, cyclosporine, tacrolimus, or other agents, in combination with long-term corticosteroids (Dall'Era, 2017). These patients remain at risk for the development of end-stage renal disease, in addition to complications from continued treatment with immunosuppressive agents.

### Immunoproteasome Inhibitors

The proteasome has been posited as a target for drug development in chronic inflammatory conditions and autoimmune disorders (Elliott, Zollner, & Boehncke, 2003). Bortezomib blocks cytokine release from immune effector cells and has demonstrated anti-inflammatory activity in several animal models of autoimmune disorders including rheumatoid arthritis (RA) (Palombella et al., 1998) and SLE (Neubert et al., 2008). More recently, bortezomib was shown to have clinical activity in patients with refractory SLE and LN who had failed standard immunosuppressive therapies (Alexander et al., 2015; de Groot et al., 2015; Zhang et al., 2017). However, systemic toxicities associated with dual-targeting proteasome inhibition, such as anemia and thrombocytopenia, restrict chronic administration (Bross et al., 2004). Further, bortezomib is associated with a dose limiting side effect of peripheral neuropathy, likely caused by off-target inhibition of the serine protease HtrA2 in neurons (Arastu-Kapur et al., 2011). Peripheral neuropathy is not induced by the peptide ketoepoxide proteasome inhibitor, carfilzomib (Arastu-Kapur et al., 2011; Dimopoulos et al., 2016).

The discovery of ONX 0914, a selective immunoproteasome inhibitor, demonstrated that the immunomodulatory and anti-inflammatory effects of dual-targeting proteasome inhibitors are due to inhibition of immunoproteasome activity in immune effector cells and inflamed tissues (Ichikawa et al., 2012; Muchamuel et al., 2009). ONX 0914 is a tripeptide ketoepoxide analog of carfilzomib and selectively inhibits the immunoproteasome in vitro and upon administration to mice. ONX 0914 exposure inhibited cytokine production in immune effector cells, reduced the number and activity of inflammatory T-cell subsets such as Th1 and Th17, increased the number of regulatory T-cells (Treg), and blocked autoantibody formation (Ichikawa et al., 2012; Kalim, Basler, Kirk, & Groettrup, 2012), (Muchamuel et al., 2009). In mouse models of RA, ONX 0914 was found to prevent joint-specific inflammation, reduce cytokine production, and ameliorate joint damage at doses one tenth of the maximum tolerated dose (MTD) (Muchamuel et al., 2009). Treatment of mice with ONX 0914 did not reduce the number of splenic lymphocytes or impair viral clearance in multiple infection models (Muchamuel et al., 2009; Mundt, Engelhardt, Kirk, Groettrup, & Basler, 2016). In addition, ONX 0914 was shown to be therapeutically active in mouse models of multiple sclerosis and SLE, in which it demonstrated equivalent activity but better tolerability than bortezomib (Basler et al., 2014; Ichikawa et al., 2012).

Immunoproteasome inhibitors contemplated in the disclosed methods include those as described in WO 07/149512 (e.g., ONX 0914), WO 96/13266 (e.g., bortezomib VELCADE®), and WO 14/152134, the disclosures of which are each incorporated by reference in their entirety. Some specific immunoproteasome inhibitors contemplated include those having a structure of formula (I):

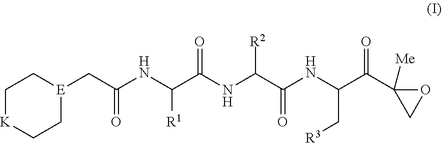

wherein
K is CH(OH) or 0;
E is N or CH;
R¹ is CH₃, CH₂OH, CH(OH)CH₃, or CH₂CN;

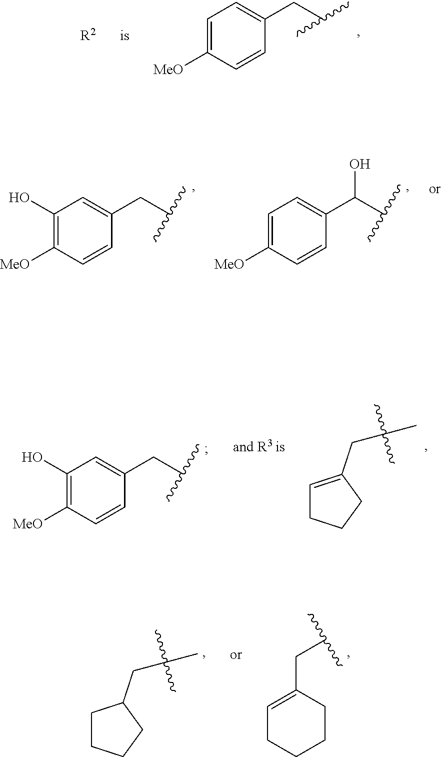

or a pharmaceutically acceptable salt thereof. In more specific embodiments, the compound of Formula (I) can have the stereochemistry of Formula (I′):

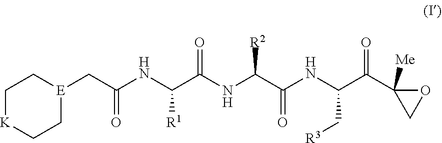

In various cases, the immunoproteasome inhibitor can be a compound having a structure as shown below:

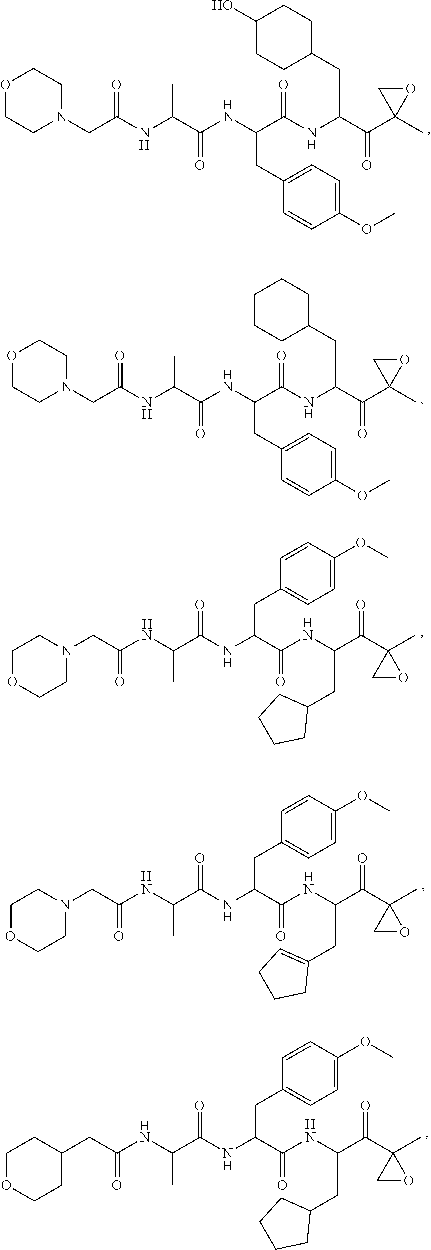

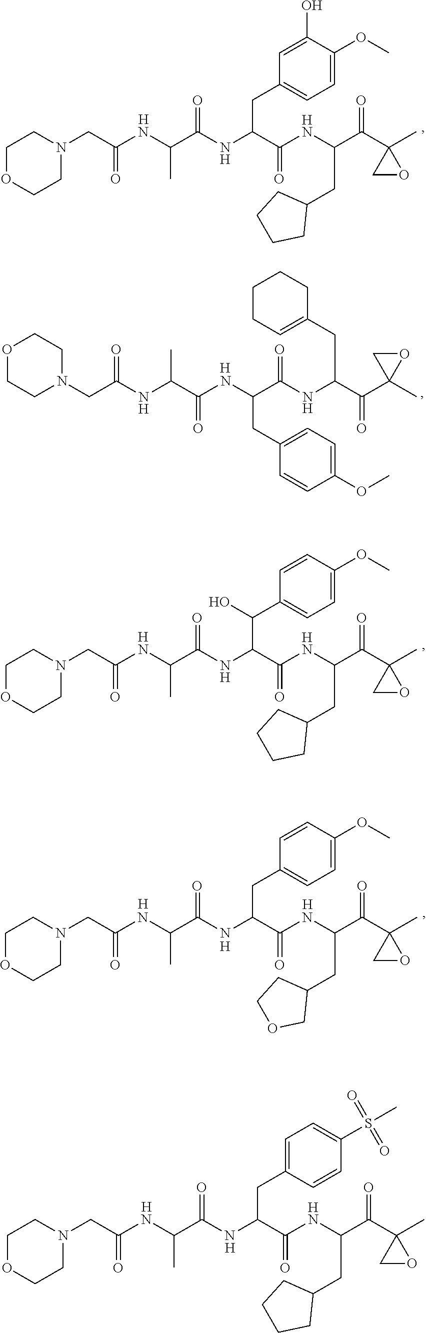

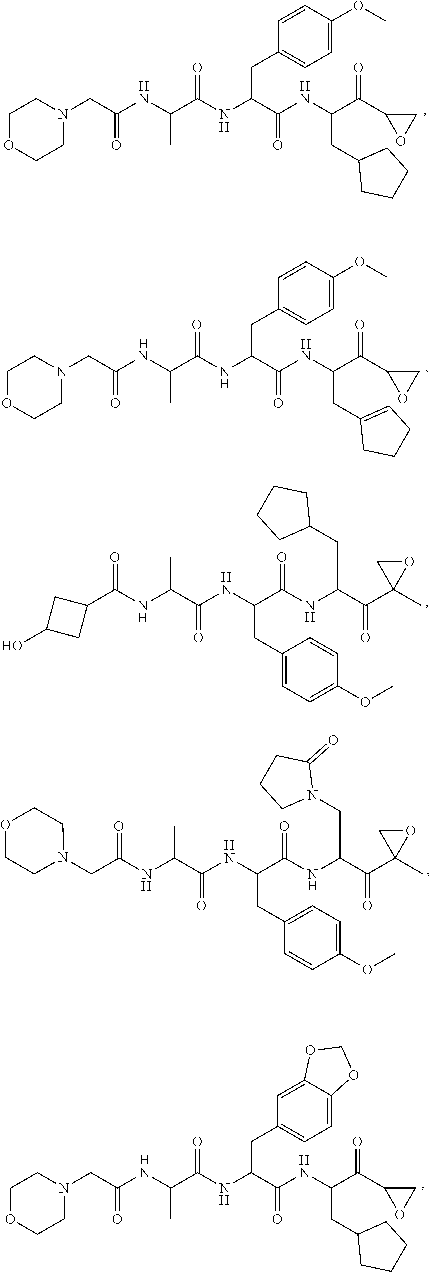

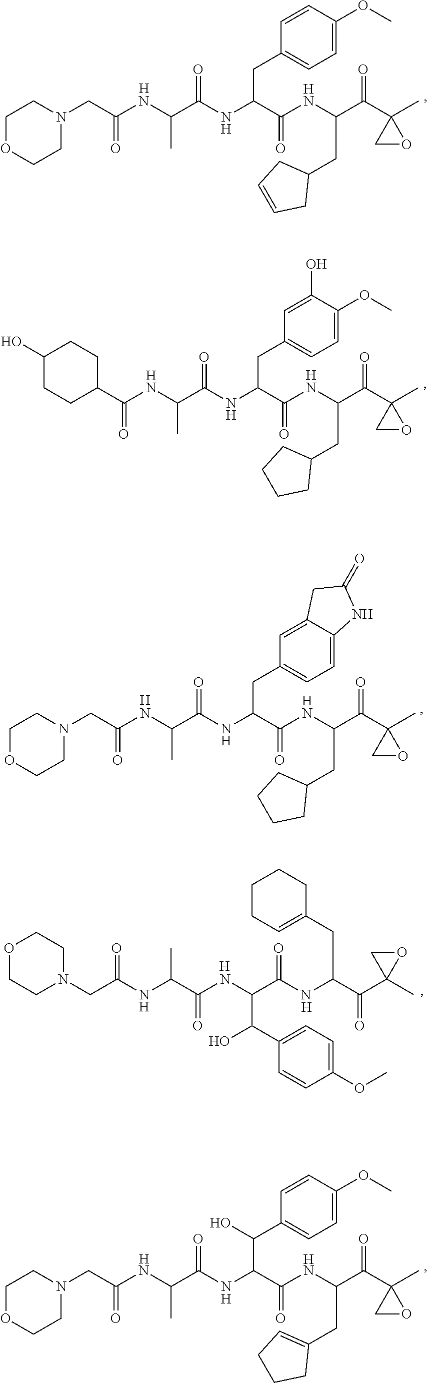

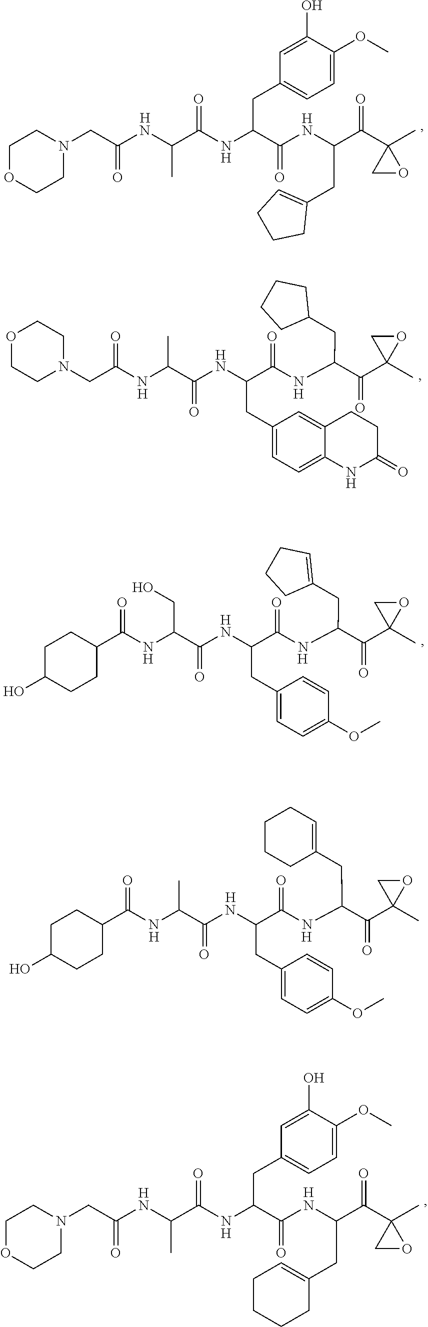

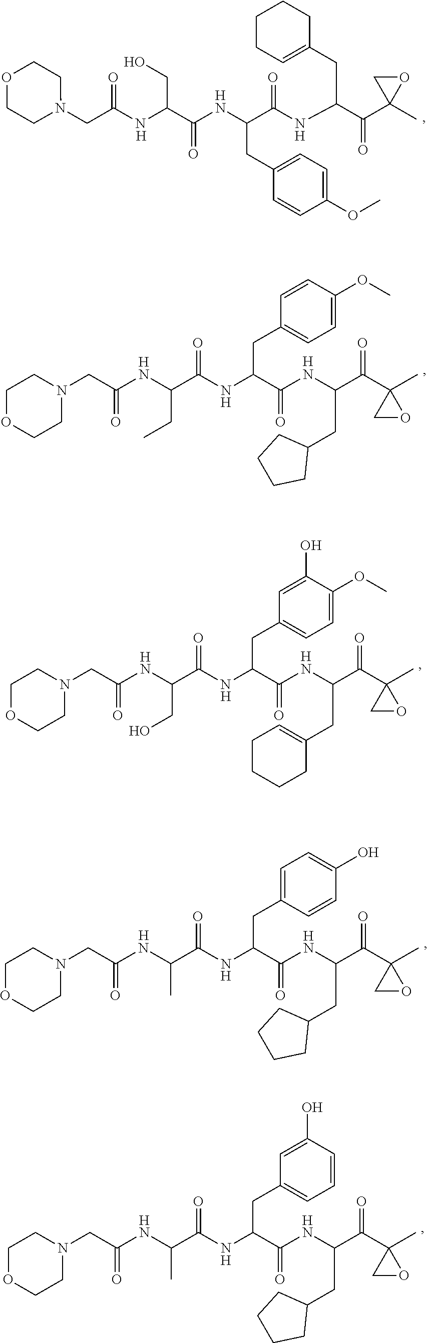

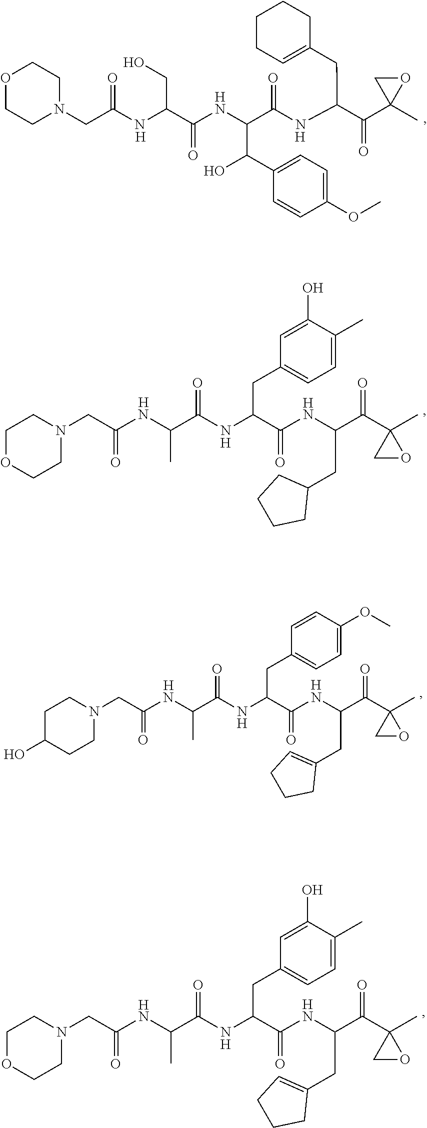

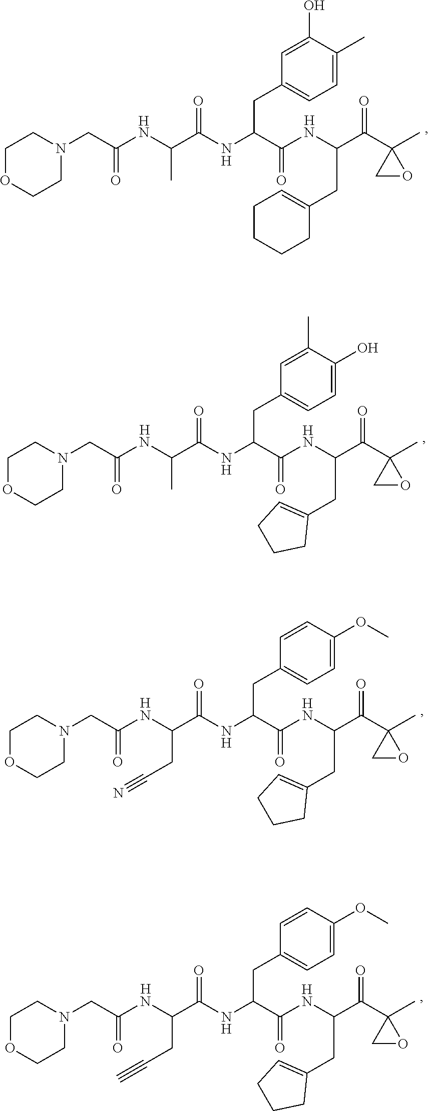

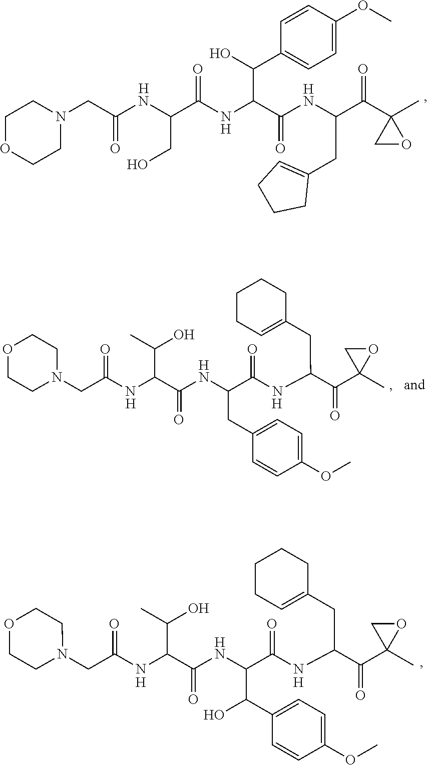

or a pharmaceutically acceptable salt thereof.

Specifically contemplated is an immunoproteasome inhibitor having a structure

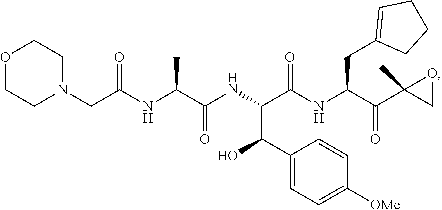 [SMILES: COc1ccc([C@@H](O)[C@H](NC(=O)[C@H](C)NC(=O)CN2CCOCC2)C(=O)N[C@@H](CC2=CCCC2)C(=O)[C@@]2(C)CO2)cc1]

or a pharmaceutically acceptable salt thereof. This compound is alternatively referred to throughout as KZR-616.

KZR-616 induces potent and selective inhibition of the immunoproteasome in human cells in vitro and potent and selective inhibition in blood and tissues when administered to rats and monkeys. KZR-616 did not inhibit any non-proteasomal targets in a broad diversity panel of biochemical assays that included 110 receptor/ligand and enzyme assays.

In vitro, KZR-616 demonstrates potent and selective inhibition (relative to(35) of the LMP7 subunit of the immunoproteasome and can target multiple subunits of the immunoproteasome at therapeutically relevant concentrations. Inhibition of immunoproteasome subunits by KZR-616 occurs through an irreversible mechanism, similar to that for both carfilzomib and ONX 0914 (Bennett & Kirk, 2008; Huber et al., 2012). In vitro, KZR-616 blocks cytokine production across multiple immune cell types, reduces the activity of inflammatory T-helper cell subsets, increases the number of regulatory T-cells, and blocks plasma cell formation and autoantibody production.

KZR-616 can be administered once weekly (e.g., every seven days) up to once bi-monthly (e.g., every 15 days), e.g., once every 7 days, once every 8 days, once every 9 days, once every 10 days, once every 11 days, once every 12 days, once every 13 days, once every 14 days, or once every 15 days. The dose of KZR-616 can be 1 to 300 mg/day. If the dose frequency is less than once daily (e.g., every 7 days), the total dose given to the subject will be multiplied by that amount e.g., 7 to 2100 mg given once every 7 days. In some cases, the KZR-616 dose is 40 to 120 mg/day (which can also be given in less than daily dosing frequency). Thus, the daily dose of KZR-616 does not indicate that the amount is given daily, but could be combined with other daily doses to be administered to the subject in less frequent doses.

The immunoproteasome inhibitor can be administered orally, subcutaneously, topically, or intravenously. In some specific cases, the immunoproteasome inhibitor is administered subcutaneously.

### Immunosuppressive Agents

The combination therapy methods disclosed herein include use of an immunosuppressive agent. As used herein, “immunosuppressive agent” refers to a substance that acts to suppress or mask the immune system of the subject being treated herein. As such, substances that suppress cytokine production, down-regulate or suppress self-antigen expression, or mask the MHC antigens are contemplated. Examples of such agents include corticosteriods, anti-miotic agents, cytokine antagonists, B-cell depleting agents, nonsteriodal anti-inflammatory agents, and an antimalarial agents.

Immunosuppressive agents contemplated include 5-amino-6-aryl-5-substituted pyrimidines (see U.S. Pat. No. 4,665,077); nonsteroidal anti-inflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, and dexamethasone; dihydrofolate reductase inhibitors such as methotrexate (oral or subcutaneous); hydroxycloroquine; sulfasalazine; leflunomide; cytokine or cytokine receptor antagonists including anti-interferon-alpha, -beta, or -gamma antibodies, anti-tumor necrosis factor-alpha antibodies (infliximab or adalimumab), anti-TNF-alpha immunoahesin (etanercept), anti-tumor necrosis factor-beta antibodies, anti-interleukin-2 antibodies and anti-IL-2 receptor antibodies; anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies: soluble peptide containing a LFA-3 binding domain (WO 90/08187 published Jul. 26, 1990); streptokinase; TGF-beta; streptodornase; RNA or DNA from the host; FK506: RS-61443; deoxyspergualin; rapamycin; T-cell receptor (Cohen et al., U.S. Pat. No. 5,114,721); T-cell receptor fragments (Offner et al., Science, 251: 430-432 (1991); WO 90/11294; laneway, Nature, 341: 482 (1989); and WO 91/01133); and T cell receptor antibodies (EP 340,109) such as T10B9.

In some cases, the immunosuppressive agent is one or more of aspirin, prednisone, methylprednisolone, sulfasalazine, leflunomide, hydroxychloroquine, belimumab, mycophenolate mofetil, mycophenolic acid, azathioprine, rituximab, ocrezilumab, entanercept, adalimumab, tocilizumab, tofacitinib, baracitinib, cyclosporine, cyclophosphamide, and tacrolimus.

In some cases, the immunosuppressive agent comprises mycophenolate mofetil, mycophenolic acid, or a pharmaceutically acceptable salt thereof. The mycophenolate mofetil, mycophenolic acid, or a pharmaceutically acceptable salt thereof can be administered in an amount of 500 mg to 3 g per day or 700 mg to 1500 mg, based upon the weight of the mycophenolate mofetil or mycophenolic acid. In some cases the immunosuppressive agent is administered once or twice daily.

In some cases, the immunosuppressive agent comprises hydroxychloroquine, azathioprine, or cyclophosphamide, or a pharmaceutically acceptable salt thereof. The hydroxychloroquine, or pharmaceutically acceptable salt thereof, can be administered in an amount of 150 to 325 mg per day, based upon hydroxychloroquine weight. The azathioprine, or pharmaceutically acceptable salt thereof, can be administered in an amount of 1 to 4 mg/kg per day, based upon azathioprine weight. The cyclophosphamide, or pharmaceutically acceptable salt thereof, can be administered in an amount of 500 to 1000 mg/m² every two to four weeks, based upon cyclophosphamide weight.

The immunosuppressive agent can be administered orally, subcutaneously, topically, or intravenously.

### EXAMPLES

NZB/W Fl mice were purchased from Jackson Laboratories. All mice were housed in the animal facility at Kezar Life Sciences. All experiment protocols were reviewed and approved by the Kezar Committee on Animal Resources. NZB/WF1 mice with established nephritis (24 weeks of age with duratble proteinuria ≥1+ proteinuria) were treated with vehicle alone, 2.5 mg/kg KZR-616 SC QW, 30 mg/kg QDx7 PO MMF or the combination of 2.5 mg/kg KZR-616 SC QW KZR-616 and 30 mg/kg QDx7 PO MMF. Proteinuria was monitored weekly once with urine dipsticks (Uristix by Bayer) and survival was observed.

To investigate immunoproteasome inhibition combined with the standard of care treatment MMF, NZB/w mice were administered vehicle alone, 2.5 mg/kg KZR-616 SC QW, 30 mg/kg QDx7 PO MMF or the combination of KZR-616 and MMF. In comparison with the untreated mice, 2.5 mg/kg KZR-616 or 30 mg/kg MMF treatment significantly reduced the level of proteinuria and increased survival. KZR-616 in combination with MMF showed significantly greater disease inhibition (as measured by proteinuria) and prolonged survival compared to vehicle or KZR-616 and MMF treatment alone.

### REFERENCES

Alexander et al., Ann Rheum Dis., 2015; 74:1474-1478.
Arastu-Kapur et al., Clin Cancer Res., 2011; 17(9): 2734-2743.
Basler et al., EMBO Molec Med., 2014; 6(2): 226-238.
Bertsias et al., Ann Rheum Dis., 2012; 71: 1771-1782.
Bross et al., Clin Cancer Res., 2004; 10:3954-3964.
Chen et al., Clin J Am Soc Nephrol., 2008; 3(1): 46-53.
Dall'era. Curr Opin Rheumatol., 2017; 29: 241-247.
De Groot, et al., Lupus Science & Med., 2015; 2(1):e000121.
Dimopoulos et al., Lancet. 2016; 17(1):27-38.
Elliott et al., J Mol Med., 2003; 81: 235-245.
EMA Guideline on clinical investigation of medicinal products for the treatment of systemic lupus erythematosus and lupus nephritis 2015.

Feldman et al., Arthritis Rheum. 2013 March; 65(3): 753-763.
Fernandez et al., Arthritis & Rheumatism (Arthritis Care & Research), 2007; 57(4):576-584.
Hahn. Ann Rheum Dis., 2011; 70(Supp 1): i64-i66.
Helmick et al., Arthritis & Rheumatism, 2008; 58(1): 15-25.
Hochbert, Arthritis & Rheumatism, 1997; 40(9): 1725.
Ichikawa, et al., Arthritis & Rheumatism, 2012; 64(2): 493-503.
Kalim et al., J Immunol., 2012; 189:4182-4193.
Kaul et al., Nature Rev. Disease Primer, 2016; 2:1-21.
Lamore et al., P & T.; 2012; 37(4):212-226.
Muchamuel et al., Nature Medicine. 2009; 15(7): 781-787.
Mundt et al., Sci. Rep., 2016; 6: 1-15.
Rovin et al., Arthritis & Rheumatism, 2012; 64(4): 1215-1226.
Seligman et al., Am J Med., 2002; 112: 726-729.
Tsokos, New Eng. J Med., 2011; 365: 2110-2121.
Weening, et al., J Am Soc Nephrol., 2004; 15: 241-250.
Wofsy, et al., Arthritis & Rheumatism, 2012; 64(11): 3660-3665.
Zhang et al., Lupus, 2017; 0, 1-7.

What is claimed:

1. A method of treating a subject suffering from an autoimmune disease comprising administering to the subject (a) an immunoproteasome inhibitor and (b) an immunosuppressive agent in an amount sufficient to treat the autoimmune disease.

2. The method of claim 1, wherein the immunoproteasome inhibitor has a structure of formula (I): 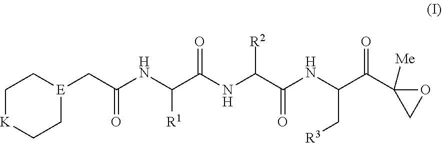
wherein
K is CH(OH) or O;
E is N or CH;
R¹ is CH₃, CH₂OH, CH(OH)CH₃, or CH₂CN; 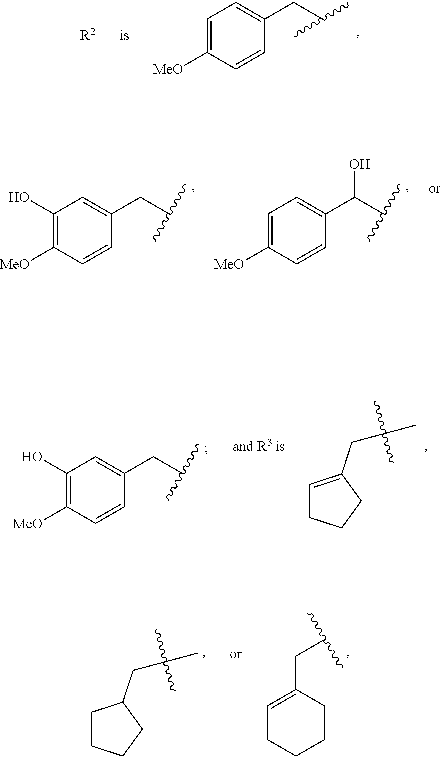
or a pharmaceutically acceptable salt thereof.

3. The method of claim 2, wherein the immunoproteasome inhibitor has a structure of 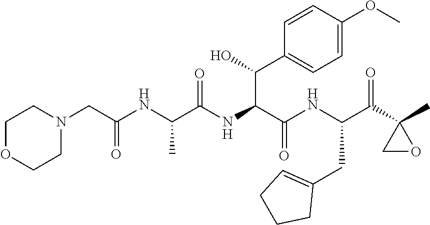 [SMILES: COc1ccc([C@@H](O)[C@H](NC(=O)[C@H](C)NC(=O)CN2CCOCC2)C(=O)N[C@@H](CC2=CCCC2)C(=O)[C@@]2(C)CO2)cc1]
or a pharmaceutically acceptable salt thereof.

4. The method of any one of claims 1 to 3, wherein the immunoproteasome inhibitor is administered in an amount of 1-300 mg per day.

5. The method of claim 4, wherein the immunoproteasome inhibitor is administered in an amount of 40-120 mg per day.

6. The method of any one of claims 1 to 5, wherein the immunoproteasome inhibitor is administered orally, subcutaneously, topically, or intravenously.

7. The method of claim 6, wherein the immunoproteasome inhibitor is administered subcutaneously.

8. The method of any one of claims 1 to 7, wherein the immunoproteasome inhibitor is administered once every 7 to 15 days.

9. The method of claim 8, wherein the immunoproteasome inhibitor is administered once every 7 days.

10. The method of any one of claims 1 to 9, wherein the immunosuppressive agent comprises a corticosteriod, an anti-miotic agent, a cytokine antagonist, a B-cell depleting agent, a nonsteriodal anti-inflammatory agent, or an antimalarial agent.

11. The method of any one of claims 1 to 10, wherein the immunosuppressive agent comprises one or more of aspirin, prednisone, methylprednisolone, sulfasalazine, leflunomide, hydroxychloroquine, belimumab, mycophenolate mofetil, mycophenolic acid, azathioprine, rituximab, ocrezilumab, entanercept, adalimumab, tocilizumab, tofacitinib, baracitinib, cyclosporine, cyclophosphamide, and tacrolimus.

12. The method of claim 11, wherein the immunosuppressive agent comprises mycophenolate mofetil, mycophenolic acid, or a pharmaceutically acceptable salt thereof.

13. The method of claim 12, wherein the mycophenolate mofetil, or pharmaceutically acceptable salt thereof, is administered in an amount of 0.5-3 g per day, based upon mycophenolate mofetil weight.

14. The method of claim 12, wherein the mycophenolic acid, or pharmaceutically acceptable slat thereof, is administered in an amount of 700 mg to 1500 mg per day, based upon mycophenolic acid weight.

15. The method of any one of claims 12 to 14, wherein the immunosuppressive is administered once per day or twice per day.

16. The method of claim 11, wherein the immunosuppressive agent is hydroxychloroquine, azathioprine, or cyclophosphamide, or a pharmaceutically acceptable salt thereof.

17. The method of claim 16, wherein the hydroxychloroquine, or pharmaceutically acceptable salt thereof, is administered in an amount of 150 to 325 mg per day, based upon hydroxychloroquine weight.

18. The method of claim 16, wherein the azathioprine, or pharmaceutically acceptable salt thereof, is administered in an amount of 1 to 4 mg/kg per day, based upon azathioprine weight.

19. The method of claim 16, wherein the cyclophosphamide, or pharmaceutically acceptable salt thereof, is administered in an amount of 500 to 1000 mg/m² every two to four weeks, based upon cyclophosphamide weight.

20. The method of any one of claims 1 to 19, wherein the immunosuppressive agent is administered orally, subcutaneously, topically, or intravenously.

21. The method of any one of claims 1 to 20, wherein the immunoproteasome inhibitor and the immunosuppressive agent are administered simultaneously.

22. The method of claim 21, wherein the immunoproteasome inhibitor and the immunosuppressive agent are co-formulated.

23. The method of any one of claims 1 to 20, wherein the immunoproteasome inhibitor and the immunosuppressive agent are administered sequentially.

24. The method of claim 23, wherein the immunoproteasome inhibitor is administered before the immunosuppressive agent.

25. The method of claim 23, wherein the immunoproteasome inhibitor is administered after the immunosuppressive agent.

26. The method of any one of claims 1 to 25, wherein the autoimmune disease is lupus nephritis or systemic lupus erythematosus (SLE).

27. The method of claim 26, wherein the autoimmune disease is SLE.

28. The method of claim 26, wherein the autoimmune disease is lupus nephritis.

29. The method of any one of claims 1 to 25, wherein the autoimmune disease is systemic vasculitis or an idiopathic inflammatory myopathy.

30. The method of any one of claims 1 to 29, wherein the subject is human.

31. The method of any one of claims 1 to 30, wherein the efficacy of administering the immunoproteasome inhibitor and the immunosuppressive agent is greater than the efficacy of administering the immunoproteasome inhibitor or the immunosuppressive agent alone.

32. The method of any one of claims 1 to 31, wherein the efficacy is exhibited by a decrease in proteinuria or urine protein to creatinine ratio compared to either (a) a subject not administered the immunoproteasome inhibitor and the immunosuppressive agent or (b) the same subject prior to administration of the immunoproteasome inhibitor and the immunosuppressive agent.

33. The method of claim 31 or 32, wherein the subject exhibits a decrease in the urine protein to creatinine ratio of at least 50% compared to the urine protein to creatinine ratio of the subject prior to administration of the immunoproteasome inhibitor and the immunosuppressive agent.

34. The method of any one of claims 31 to 33 , wherein the subject exhibits a urine protein to creatinine ratio of 0.5 or less after administration of the immunoproteasome inhibitor and the immunosuppressive agent.
